(19) United States
(12) Patent Application Publication
(10) Pub. No.: US 2009/0148558 A1
(43) Pub. Date: Jun. 11, 2009

(54) METHOD OF PRODUCING MUSHROOM MYCELIA BASED MEAT ANALOG, MEAT ANALOG PRODUCED THEREBY, LOW CALORIE SYNTHETIC MEAT, MEAT FLAVOR AND MEAT FLAVOR ENHANCER COMPRISING THE MEAT ANALOG

(75) Inventors: Young-Duk Kim, Seoul (KR); Yong-Hwi Kim, Seoul (KR)

Correspondence Address: CANTOR COLBURN, LLP, 20 Church Street, 22nd Floor, Hartford, CT, 06103, US

(73) Assignee: CJ CORP., Seoul (KR)

(21) Appl. No.: 12/302,453

(22) PCT Filed: May 25, 2007
(86) PCT No.: PCT/KR2007/002553

§ 371 Date: Nov. 25, 2008

(30) Foreign Application Priority Data
May 25, 2006 (KR) 10-2006-0047116

(51) Int. Cl. A23L 1/31; C12N 1/16; A23L 1/231

(52) U.S. Cl. 426/49; 426/574; 426/534

(57) ABSTRACT

Provided is a method of producing mushroom mycelia-based meat analog, a meat analog produced using the method, a low-calorie synthetic meat and a meat flavor comprising the meat analog. The meat analog can be produced from mushroom mycelia within a short period of time in a cost and effort effective manner. A meat analog having improved meat-like texture and flavor compared to a conventional soy protein can be produced, and thus a low-calorie synthetic meat and a meat flavor can be produced using the meat analog.

### TECHNICAL FIELD

The present invention relates to a mushroom mycelia-based meat analog which is one kind of plant protein, and more particularly, to a method of producing a meat analog by mass-producing mushroom mycelia, a meat analog produced thereby, and a low-calorie synthetic meat and a meat flavor using the meat analog.

### BACKGROUND ART

Meat analog products were originally derived to be used for a wide variety of vegetarian food products. Recently, however, an increasing number of non-vegetarians are willing to pay more for safe and eco-friendly clean foods with growing health and nutrition concerns such as chronic disease prevention and thus the market for the meat analog products is expected to be expanded. Accordingly, novel food materials and food production technology for meat analogs are required to be developed.

For the manufacture of a meat analog product, health and nutritional values of the meat analog such as calorie and fat content as well as physiological reactions and safety of the meat analog are required to be considered. Further, flavor and texture of meat are required to be imitated. Particularly, the most important properties of the meat analogs to be developed are texture similar to that of meat and natural in-mouth feeling and juiciness of meat resulting from moisture and fat that are retained in meat.

Such meat analogs are developed mainly from soy proteins. Although, the meat analogs made from the soy proteins may have meat-like structure and appearance, the natural meat texture cannot be completely reproduced by the soy proteins. Thus, the meat analogs made from soy proteins have only been used in a sliced or minced meat form, usually for burgers and meatballs. A gel strength of soy proteins which can form a meat-like texture may vary according to soy protein fractionation. Typically, soy protein-based products having a fibrous structure imitating texture of meat have been developed using wheat gluten. However, since the soy protein has bitterness including beany flavor, or the wheat gluten sometimes causes allergies in people, the use of them has been limited. Further, meat analogs made from legume or grain, based on soy protein or wheat gluten cannot completely imitate meat-like texture, and nor have meat-like flavor. Therefore, novel food materials for the meat analogs are required to be developed.

Studies on use of a mycoprotein, as food, which is made by converting carbohydrates produced by aerotropic microfunge living in soil to proteins, have been conducted since the 1960s (Sadler, M. J., Myco-protein, in Encyclopedia of Food Sci., Food Technol., Nutr., 1993, pp. 3191-3196, R. Macrae et al. (ed.), Academic Press). However, only a meat analog called “Quorn” has been successfully used for food so far. “Quorn” is a fungal meat analog produced using Fusarium graminearum by Quorn located in the UK since 1964, and safety thereof was verified in 1984 (Trinci, A. P. J., Evolution of the Quorn Registered myco-protein fungus, Fusarium graminearum A3/5. Microbiology, 140(9), pp. 2181-2188, 1994). However, possibilities of food allergy induced by the fungal meat analog and negative perceptions against fungi have limited the market for the meat analogs using the same.

Accordingly, mushroom having a fibrous structure and meat-like flavor have been focused on as a novel food material for meat analogs. While mushrooms have relatively low protein content compared to soy protein, mushrooms can effectively improve in-mouth feeling and flavor of meat analogs since they have high sulfur-containing amino acid content and glutamic acid content. Further, meat-like texture can be more effectively reproduced using mushrooms compared to soy proteins since mushrooms are composed of fibrous carbohydrates. Unlike negative perceptions against fungi mushrooms have been widely used since ancient times, and recently, it has been widely known that mushrooms have various physiologically functional materials. Thus, it is expected that a meat analog using mushroom-based mycoprotein has more potential than soy protein-based or fungal mycoprotein.

However, since mushrooms, which in Korea cost 7,000 to 8,000 Korean won per kg, are expensive and mushroom cultivation takes 20 to 30 days, mushroom-based meat analog cannot be easily commercialized by mass-production. Therefore, it is required to develop a technology to produce a large amount of mushroom mycelia with relatively low costs, so that the meat analogs and various products such as a meat substitute, a meat flavor and meat flavor enhancer can be developed based on the mushroom mycelia.

### DISCLOSURE OF THE INVENTION

The present invention provides a method of producing a meat analog from mushroom mycelia by preparing optimum mediums and conditions for culturing mushroom mycelia.

The present invention also provides a meat analog produced using the method and a synthetic meat based on the meat analog having improved meat-like texture and flavor compared to a conventional soy protein.

The present invention also provides a meat flavor and meat flavor enhancer using the meat analog produced using the method to decrease the amount of conventionally used hydrolysated vegetable protein (HVP) and chemical seasonings such as monosodium glutamate by 30 to 40%.

According to an aspect of the present invention, there is provided a method of producing a meat analog, the method including: producing mushroom mycelia; mixing the mushroom mycelia with a protein complement and a binding agent; and texturizing the mixture into a protein form by extruding the mixture prepared in the mixing, wherein the producing of the mushroom mycelia comprises culturing mushroom mycelia or spores in a liquid medium comprising sugar cane extract.

The mushroom mycelia and spores cultured in the liquid medium may be mycelia and spores of edible mushrooms such as Pleurotus ostreatus, Agaricus bisporus, Flammulina velutipes, Pleurotus eryngii, or functional mushrooms such as Ganoderma mushroom and Cordyceps.

The mushroom mycelia or spores cultured in the liquid medium may be preferably Agaricus bisporus mycelia or spores.

In addition, the liquid medium may include 10 to 30 g/l of sugar cane extract.

The liquid medium used herein further includes sodium nitrate as a nitrogen source.

The concentration of the sodium nitrate in the liquid medium may be in the range of 1 to 10 g/l.

Further, the liquid medium may further include 1 to 10 g/l of yeast extract.

The present invention also provides a meat analog produced using the method, a low-calorie synthetic meat including the meat analog as a main ingredient, and a meat flavor and meat flavor enhancer employing the meat analog.

The mushroom mycelia according to the method of an embodiment of the present invention is produced using a typical liquid culture method including: isolating a mushroom strain of interest, inoculating the strain of interest into an optimum medium, and culturing the medium to prepare a seed culture; preparing an inoculum from the prepared mushroom seed culture; and main-culturing the inoculum to mass-produce mushroom mycelia.

First, the primary strain of interest is isolated from tissues or spores of mushroom fruit body. Typically, the primary strain is a strain from which mushroom seed grows when the mushroom seed is prepared. The mushroom seed which functions like the seed of crops refers to a pure culture of a desired mushroom strain prepared by cultivation. The inoculum, which is a strain inoculated into a culture medium, serves as an intermediate mushroom seed for proliferation since a sufficiently large number of mushroom seeds cannot be directly cultured from the primary strain.

Meanwhile, a culture medium or a culture bottle refers to a substrate that supports the growth of mycelia, with nutrients and controlled pH based on processed rice straw, compost, malt, potato, and the like is. To isolate or mass-produce mushroom mycelia, a culture medium optimized for physiological characteristics of the desired mushroom mycelia is required. The culture medium provides nutrition sources and moisture required for growth of mushroom mycelia, and thus essential elements for the life of the mushroom mycelia such as a carbon source, a nitrogen source, vitamins, minerals are needed to be included in the culture medium.

In the preparing the mushroom seed, a medium that is commonly used for culturing mushroom, such as a potato dextrose agar medium, a yeast extract malt extract glucose medium, and the like can be used as the medium. The purpose of preparing the mushroom seed is to produce a large amount of seed using purely cultured strains under microbiologically stable conditions.

Next, a large number of mushroom seeds must be simultaneously used to mass-produce mushroom mycelia. However, culturing the mushroom seeds from the primary strain is a time and effort consuming process. Thus, an inoculum is prepared as an intermediate culture by pre-culturing the mushroom seed. The preculture medium may include 15 to 25 g/l of potato dextrose broth, 10 g/l of yeast extract, 2 to 5 g/l of malt extract, and 2 to 5 g/l of soytone, and preferably include 24 g/l of potato dextrose broth, 10 g/l of yeast extract, 5 g/l of malt extract, and 5 g/l of soytone. The preculture may be performed for about 3 to 4 days.

Finally, the inoculum obtained in the preculture process is proliferated in the main culture process. In the main culture, a mixture of the liquid medium and the mushroom strain is stirred by injecting filtered pressed air therein to uniformly contact the mycelia of the strain with nutrients, and to raise oxygen content in the stationary liquid phase in which the oxygen content is usually not sufficient.

In the main culture, sugar cane extract may be used as the carbon source and soytone may be used as the nitrogen source in the medium when considering mycelia growth, whereas sodium nitrate may be preferable as the nitrogen source when considering cost-effectiveness as well as the mycelia growth. The sugar cane extract refers to unrefined extract sugar prepared by extracting the sugar cane juice and concentrating and crystallizing the sugar cane juice. The sugar cane extract provides growth factors in addition to carbon in this invention. When the concentration of sugar cane extract is less than 10 g/l, the mushroom mycelia cannot grow well. On the other hand, when the concentration of sugar cane extract is greater than 30 g/l, osmotic pressure is too high and the process is cost-ineffective. Thus, the concentration of the sugar cane extract may be in the range of 10 to 30 g/l. Sodium nitrate as an inorganic nitrogen source is converted to a high value-added organic nitrogen by mushroom mycelia. Further, although other ammonia based nitrogen sources may change the pH of the medium, sodium nitrate as the nitrogen source can control the pH of the medium, and thus the pH of the medium can be constantly maintained. When the concentration of the sodium nitrate is less than 1 g/l, the amount of the nitrogen source required for the mycelia growth may not be sufficient. On the other hand, when the concentration of the sodium nitrate is greater than 10 g/l, there is no corresponding remarkable growth of mycelia, thus the concentration of the sodium nitrate may be in the range of 1 to 10 g/l. Further, addition of 1 to 10 g/l of yeast extract may be more for the growth of Agaricus bisporus mycelia. Particularly, the main culture medium may include 15 to 25 g/l of sugar cane extract, 5 to 10 g/l sodium nitrate, and 5 to 10 g/l of yeast extract. The remaining portion of the preculture medium and the main culture medium is filled with distilled water or sterilized drinking water.

Typically, the mushroom mycelia can grow at a temperature in the range of 3 to 30° C., and particularly, an optimum temperature and pH for the growth of Agaricus bisporus mycelia is respectively in the range of 24 to 25° C. and pH 6.8 to 7.0. The temperature for the preculture and the main culture may be in the range of 28 to 30° C., and preferably 28° C. The highest growth of mycelia may be found when an initial pH is in the range of 6.0 to 6.5 in the preculture and the main culture according to an embodiment of the present invention.

According to an embodiment of the invention, the main culture may be performed for 3 to 10 days. When the main culture is performed for less than 3 days, the mycelia may not sufficiently grow. On the other hand, when the main culture is performed for longer than 10 days, the culture process may not be cost ineffective. To maximize yield and economical efficiency, the main culture may preferably be performed for 3 to 6 days, which is far shorter than 14 to 15 days typically taken for a liquid culture of mushroom mycelia in prior arts.

In conventional cultures, a homogenizer has been used to uniformly distribute the mushroom mycelia particles in the medium before inoculating the mushroom seed into the medium. However, the mushroom mycelia particles may become too small due to the homogenization, thereby making the conventional cultures inefficient. Thus, the mushroom mycelia may be uniformly distributed using a blender in the preculture and main culture according to an embodiment of the present invention.

Further, the preculture and main culture may be culture with stirring or shaking in which a rotation frequency may be 200 rpm to maximize yield.

In the mixing of the mushroom mycelia produced using the method of the present invention with a protein complement and a binding agent, a conventional material for meat analog such as a hydrolyzed vegetable protein (HVP) including a soy protein, a grain protein, and the like, meat such as beef or chicken, fish, vegetables, or nuts may be used as the protein complement. Egg albumin may be used as the binding agent. Glucan, nucleotide, sulfur-containing amino acid, glutamic acid, starch, dietary fiber, and the like may be included in the mixing process in addition to the protein complement and the binding agent, and a flavor, a coloring agent, and the like can further be included.

The texturizing of the composition obtained from the mixing into a form of the protein may be performed by extruding the composition prepared in the mixing the mushroom mycelia with the protein complement and the binding agent. In the extrusion, raw material component ratio, feeding amount of the raw material, moisture content, screw rotation velocity, and barrel heating temperature can be controlled. The extrusion can be performed at a temperature in the range of 100 to 170° C. under a pressure in the range of 100 to 1000 psi with an increased moisture content of 40%. A cooling die or a circular die may be used for texturization into a protein or meat analog.

A synthetic meat according to an embodiment of the present invention may comprise the meat analog produced using the method according to the present invention, and further comprise known sub-materials such as protein, carbohydrate, fat, a flavor, and a coloring agent. Here, 10 to 100% of meat analog based on the amount of the total protein of the synthetic meat may be included.

A meat flavor and meat flavor enhancer according to an embodiment of the present invention are produced using the meat analog produced by the method of the invention by a conventional method in the art.

### BRIEF DESCRIPTION OF THE DRAWINGS

FIG. 1 is a schematic flow diagram of producing a meat analog, a synthetic meat and a meat flavor and meat flavor enhancer according to an embodiment of the present invention.

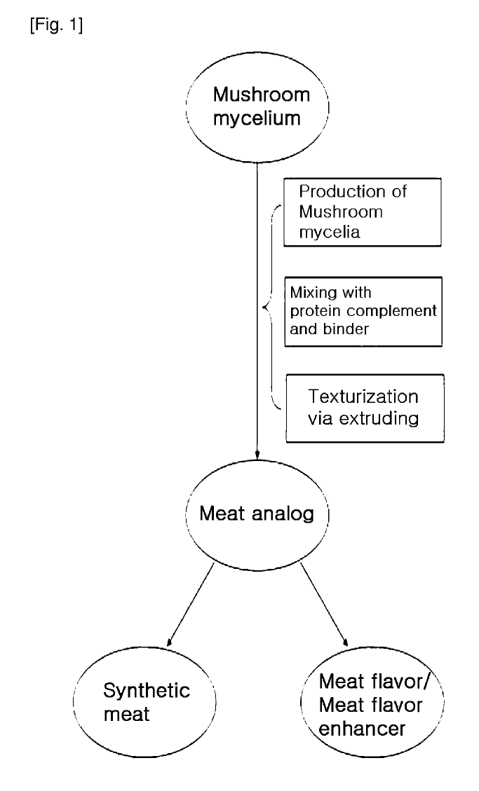

### BEST MODE FOR CARRYING OUT THE INVENTION

Hereinafter, the present invention will now be described more fully with reference to the accompanying drawings, in which an exemplary embodiment of the invention is shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

### Example 1

### Mass-Production of Mushroom Mycelia in a Liquid Culture for Meat Analogs

(1) Isolation of a Strain and Preparation of an Inoculum

A strain was obtained from tissue culture of Agaricus bisporus and it was cultured on a potato dextrose agar (PDA) medium at 25° C. for 3 weeks, and then the Agaricus bisporus strain was preserved at 4° C.

For a solid culture, an inoculum was prepared by partially separating mycelia from the center of a PDA plate medium which had been preserved in a refrigerator, and inoculating and culturing the separated mycelia in a thermostat at 25° C. For a liquid culture, an inoculum was prepared by autoclave-sterilizing 100 ml of a PDBYMS medium comprising 20 g/l of potato dextrose broth (PDB), 10 g/l of yeast extract, 5 g/l of malt extract, and 5 g/l soytone in a 500 ml Erlenmeyer flask at 121° C. for 15 minutes, inoculating a part of the mycelia into the PDBYMS medium, and culturing in a liquid culture at 25° C., while stirring at 200 rpm.

(2) Preculture of Mushroom Mycelia for Seed

To find out optimum conditions for preculture of mushroom seed, culture media were prepared in the conditions as described in Table 1: 100 ml of each of the prepared culture media was respectively placed in a 500 ml Erlenmeyer flask; the flasks were autoclave-sterilized at 121° C. for 15 minutes; 1% (v/v) of homogenized inoculum was aseptically inoculated into the culture media; and the mycelia was cultured by shaking in a thermostat at 25° C. at 200 rpm for 4 days with the growth of mycelia monitored.

TABLE 1

| | | Yeast | | | | |
|---|---|---|---|---|---|---|
| | PDB | extract | Malt extract | Soytone | Weight | Dried weight |
| | [g/l] | [g/l] | [g/l] | [g/l] | [g/100 ml] | [g/100 ml] |
| P1 | 0 | 10 | 5 | 5 | 6.49 | 0.46 |
| P2 | 5 | 10 | 5 | 5 | 7.50 | 0.65 |
| P3 | 10 | 10 | 5 | 5 | 8.35 | 0.93 |
| P4 | 15 | 10 | 5 | 5 | 7.65 | 1.01 |
| P5 | 20 | 10 | 5 | 5 | 7.95 | 1.06 |
| P6 | 24 | 10 | 5 | 5 | 8.43 | 1.53 |
| Y1 | 24 | 0 | 5 | 5 | 7.53 | 0.85 |
| Y2 | 24 | 3 | 5 | 5 | 7.07 | 0.95 |
| Y3 | 24 | 5 | 5 | 5 | 6.87 | 0.71 |
| Y4 | 24 | 10 | 5 | 5 | 8.45 | 1.14 |
| M1 | 24 | 10 | 0 | 5 | 5.88 | 0.79 |
| M2 | 24 | 10 | 0.5 | 5 | 5.91 | 0.76 |
| M3 | 24 | 10 | 1 | 5 | 6.37 | 0.7 |
| M4 | 24 | 10 | 2 | 5 | 6.66 | 0.94 |
| M5 | 24 | 10 | 5 | 5 | 6.57 | 0.67 |
| S1 | 24 | 10 | 5 | 0 | 6.50 | 0.63 |
| S2 | 24 | 10 | 5 | 0.5 | 6.72 | 0.74 |
| S3 | 24 | 10 | 5 | 1 | 6.38 | 0.74 |
| S4 | 24 | 10 | 5 | 2 | 6.58 | 0.78 |
| S5 | 24 | 10 | 5 | 5 | 6.94 | 0.71 |

The mycelia growth was measured by filtering the contents of the Erlenmeyer flask containing the mycelia using a gauze, centrifuging the filtrate at 1500 rpm for 10 minutes using a centrifugal separator, drying the separated mycelia in a dry oven for 24 hours, and measuring the weight of the dried mycelia.

As described in Table 1, the growth of the mycelia was maximized in a culture medium composed of 24 g/l of PDB, 10 g/l of yeast extract, 2 to 5 g/l of malt extract, and 2 to 5 g/l of soytone. Thus, a PDBYMS medium composed of 24 g/l of PDB, 10 g/l of yeast extract, 5 g/l of malt extract, and 5 g/l of soytone was used as a basic medium for preculture of mushroom seed.

(3) Main Culture

BIOFLO IIc Batch/Continuous Fermentor (New Brunsdwick Scientific) was used as a bioreactor.

To find out an optimum temperature for culturing mycelia, 100 ml of the basic medium was introduced into a 500 ml Erlenmeyer flask, and the flask was sterilized by autoclaving at 121° C. for 15 minutes. Then, 1% (v/v) of aseptically homogenized inoculum was inoculated into the culture medium, and cultured with shaking in a thermostat at temperatures of 25° C., 28° C., and 30° C. at 200 rpm for 4 days to measure growth of the mycelia. As a result, mycelia grew well at 28° C. and 30° C., and particularly the growth of the mycelia was maximized at 28° C.

To find out an optimum initial pH for culturing mycelia, 100 ml of the basic medium was introduced into a 500 ml Erlenmeyer flask, and the pH of the basic medium was adjusted using phosphoric acid and 50% sodium hydroxide (NaOH) to have an initial pH in the range of 4.0 to 9.0 in steps of 0.5. Then, the culture mediums were sterilized with different pH by autoclaving at 121° C. for 15 minutes, and 1% (v/v) of homogenized inoculum was aseptically inoculated into each of the culture medium and cultured with shaking in a thermostat at 25° C. at 200 rpm for 4 days to measure growth of the mycelia. As a result, the growth of the mycelia was maximized at pH 6.0.

To find out the effect of culture period, 100 ml of the basic medium was introduced into a 500 ml Erlenmeyer flask, and the pH of the basic medium was adjusted to have an initial pH in the range of 6.0 to 6.5. Then, the culture medium was sterilized by autoclaving at 121° C. for 15 minutes, and 1% (v/v) of homogenized inoculum was aseptically inoculated into the culture medium and cultured with shaking in a thermostat at 25° C. at 200 rpm for 10 days to measure growth of the mycelia. As a result, the growth of mycelia exhibited a typical exponential phase in which the growth rapidly increased from the third day onwards after the mild increase for the first 2 days. The maximum amount of the mycelia was 2.43 g/100 ml in the ninth day of culturing and the growth of the mycelia declined thereafter.

To select an optimum medium for the main culture, the growth of mycelia was measured by modifying the basic medium. That is, 20 g/l of each of PDB, glucose and sugar cane extract (CJ Corporation) as a carbon source was mixed with 10 g/l of yeast extract, 5 g/l of malt extract, and 5 g/l of soytone, and the pH of the mixture was adjusted to a pH of 6.0 to 6.5. Then, 100 ml of the medium was introduced into a 500 ml Erlenmeyer flask, and the culture medium was sterilized by autoclaving at 121° C. for 15 minutes. 1% (v/v) of homogenized inoculum was aseptically inoculated into the culture medium and cultured while shaking in a thermostat at 25° C. at 200 rpm for 4 days to measure the growth of the mycelia. As a result, the growth efficiency of the mycelia was maximized when using sugar cane extract as a carbon source.

Then, to find out an optimum concentration of the carbon source, each of 1 g/l, 5 g/l, 10 g/l, 15 g/l, and 20 g/l of sugar cane extract was used to prepare separate mixtures with 10 g/l of yeast extract, 5 g/l of malt extract, and 5 g/l of soytone, and the ph of the mixture was adjusted to have a pH of 6.0 to 6.5. 100 ml of the medium was introduced into a 500 ml Erlenmeyer flask, the culture medium was sterilized by autoclaving at 121° C. for 15 minutes to measure growth of the mycelia in the same manner as in selecting the optimum the carbon source. As a result, the mycelia grew well when the concentration of the sugar cane extracts was 10 to 20 g/l.

To find out an optimum nitrogen source and the concentration of the nitrogen source for culturing mycelia, 20 g/l of sugar cane extract as the optimum carbon source was added to a PDBYMS medium, and 10 g/l of each of sodium nitrate, ammonium nitrate, ammonium chloride, ammonium sulfate, and soytone as a nitrogen source was further added thereto. Then, the pH of the culture medium was adjusted to have a pH of 6.0 to 6.5, 100 ml of the medium was introduced into a 500 ml Erlenmeyer flask, and the culture medium was sterilized by autoclaving at 121° C. for 15 minutes to measure growth of the mycelia in the same manner as in selecting the carbon source. As a result, the growth of the mycelia was the highest when soytone was used as the nitrogen source followed by that when sodium nitrate was used as the nitrogen source. When considering cost effect as well as the growth efficiency, sodium nitrate is the most preferred nitrogen source.

Then, to find out an optimum concentration of the nitrogen source, the growth of mycelia was measured in the same manner as in selecting the nitrogen source, except that each of 1 g/l, 3 g/l, 5 g/l, 7 g/l, and 10 g/l of sodium nitrate was used to prepare separate mixtures. As a result, the growth of the mycelia was the highest when the concentration of sodium nitrate was 10 g/l. Here, when 5 g/l of yeast extract was added thereto, the growth of mycelia was maximized. Accordingly, a culture medium composed of 20 g/l of sugar cane extract, 10 g/l of sodium nitrate, and 5 g/l of yeast extract was determined as an optimum culture medium for the main culture.

Three preparations of 1% (v/v) of an aseptically homogenized inoculum were inoculated into each of 2 l of the main culture medium, and cultured at a rotational frequency of impeller of the bioreactor of respectively 200 rpm, 250 rpm and 300 rpm with 0.25 v/v/m of air injection for 4 days to measure the weight of the mycelia. Afterwards the mycelia obtained from each preparation were freeze-dried for 7 days, and the total dry weight of each was measured. As a result, a maximum amount of the mycelia was obtained at a rotational frequency of an impeller of 200 rpm.

### Example 2

### Production of Meat Analog

Meat analog was produced using a method that is commonly used in the manufacture of plant proteins (Korean Food Research Institute, Studies on the Development and Application of Functional Food Materials Using by Myco-protein, Final Report, Ministry of Agriculture & Forestry, Nov. 18, 2002). 40% of mushroom mycelia produced in Example 1, 30% of corn hull, and 30% of egg albumin were mixed and the moisture content was adjusted to 40%. The mixture was extruded at a temperature in the range of 100 to 170° C. under a pressure in the range of 100 to 1000 psi. A cooling die was used in the extrusion to produce mushroom-based meat analog.

### Example 3

### Preparation of a Synthetic Meat from the Meat Analog

A synthetic meat was produced using a method that is commonly used in the manufacture of synthetic meats. 50 wt % of the meat analog produced in Example 2, a small amount of seasonings, flavors, coloring agents, and the like were mixed to produce a synthetic meat.

### Example 4

### Preparation of a Meat Flavor from the Meat Analog

The meat analog produced in Example 2 was pulverized, and viscera removed anchovy, washed and cut kelp and brown seaweed were prepared. 20 wt % of anchovy, 20 wt % of kelp, and 20 wt % of brown seaweed based on 100 wt % of water were boiled at 10° C. until the half of water had evaporated to obtain a broth. The anchovy, kelp, and brown seaweed were immersed in the broth for 12 hours, and they were dried and pulverized. A meat flavor was prepared by mixing 65 wt % of the pulverized meat analog, 10 wt % of the anchovy powder, 10 wt % of the kelp powder, and 5 wt % of the brown seaweed powder.

### INDUSTRIAL APPLICABILITY

According to the method of producing a mushroom mycelia-based meat analog of the present invention, a meat analog is produced by culturing mushroom mycelia using a liquid culture under predetermined media and conditions for 3 to 6 days, and thus a meat analog and a synthetic meat having excellent texture and flavor compared to conventional soy proteins and fungal proteins can be produced within a short period of time in a cost and effort effective manner. Further, the synthetic meat of the invention has stable characteristics such that additives such as flavors and coloring agents do not leak out of it in the cooking process. Further, the amount of conventionally used hydrolysated vegetable protein (HVP) and chemical seasonings such as monosodium glutamate can be reduced by 30 to 40% using the meat flavor from the meat analog.

1. A method of producing mushroom mycelia based meat analog, the method comprising:
producing mushroom mycelia;
mixing the mushroom mycelia with a protein complement and a binding agent; and
texturizing the mixture into a protein form by extruding the mixture prepared in the mixing,
wherein the producing of the mushroom mycelia comprises culturing mushroom mycelia or spores in a liquid medium comprising sugar cane extract.

2. The method of claim 1, wherein the culturing of mushroom mycelia or spores in a liquid medium is conducted 3 to 6 days before completion.

3. The method of claim 1, wherein the mushroom mycelia or spores cultured in the liquid medium is Agaricus bisporus mycelia or spores.

4. The method of claim 1, wherein the liquid medium comprises 10 to 30 g/l of sugar cane extract.

5. The method of claim 1, wherein the liquid medium further comprises sodium nitrate as a nitrogen source.

6. The method of claim 5, wherein the concentration of the sodium nitrate is in the range of 1 to 10 g/l.

7. The method of claim 1, wherein the liquid medium further comprises 1 to 10 g/l of yeast extract.

8. A meat analog produced using a method of claim 1.

9. A synthetic meat comprising the meat analog of claim 8.

10. A meat flavor comprising the meat analog of claim 8.
